(19) United States
(12) Patent Application Publication
(10) Pub. No.: US 2008/0112847 A1
(43) Pub. Date: May 15, 2008

(54) COLLECTING AND TESTING DEVICE AND METHOD OF USE

(76) Inventor: Nancy Yu Chen, Cupertino, CA (US)

Correspondence Address: UCP Biosciences, Inc.;Attn: Nancy Yu Chen, 10511 John Way,, Cupertino, CA, 95014, US

(21) Appl. No.: 11/939,867

(22) Filed: Nov. 14, 2007

Related U.S. Application Data
(60) Provisional application No. 60/858,945, filed on Nov. 15, 2006.

(51) Int. Cl. G01N 21/01; B01J 19/00; G01N 33/48

(52) U.S. Cl. 422/58; 422/68.1; 73/61.41

(57) ABSTRACT

The present invention provides a new and improved collecting and testing device comprising a specimen container for collecting and/or storing specimen, a testing chamber having at least one test element, and a conduit for automatically transferring a portion of the specimen from the specimen container to the testing chamber.

This application claims the benefit of priority of U.S. Ser. No. 60/858,945, filed Nov. 15, 2006. The entire content and disclosure of the preceding application are incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

The present invention provides collecting and testing devices comprising a specimen container, a test chamber and a conduit, and methods of use thereof.

### BACKGROUND OF THE INVENTION

A variety of collection and test devices for professional or home use are available and described in the literature. These test devices often utilize a container to collect and store the specimen. The specimen can then be transferred to a test chamber comprising at least a test element to determine the presence or absence of an analyte. These devices can be used for the detection of drugs or biological compounds such as glucose, hormones, antibodies or etiological agents. However, many of these devices are complex in design and manufacture and fabricated of relatively expensive materials. In addition, many of these devices require precise action(s) by the operators such as placing the devices in proper positions during testing, or insert a key into an appropriate location in the device to initiate reaction, or the test needs to be performed by several steps of procedures. The step(s) or procedure(s) needed during test may increase the chance of getting additional error. Furthermore, many of the previously described devices place the original specimen in the container in direct fluid communication with the specimen in the test chamber, thus it may cause potential contamination of the original specimen in the container by contacting chemical reagents from testing strips and bio-burdens in the test chamber. Thus, the original specimen may not be retested, thereby preventing confirmation testing. The present invention addresses these problems, and provides related benefits.

### SUMMARY OF THE INVENTION

The present invention describes a new and improved collecting and testing device that comprises a specimen container for collecting and/or storing specimen, a testing chamber having at least a test element such as a test strip, and a conduit for transferring a portion of the specimen from the specimen container to the testing chamber. In one embodiment, while the specimen is added into the specimen container, at least a portion of the specimen can automatically enter into the testing chamber through the conduit so that the specimen contacts a test element in the testing chamber. The conduit allows fluid transferring from the specimen container to the testing chamber. The specimen in the testing chamber is in indirect fluid communication with the specimen in the specimen container. Furthermore, the specimen stored in the container is separable from the specimen stored in the testing chamber, and the specimen in the testing chamber cannot flow back into the specimen container, so that potential contamination of the original specimen by reagent testing strips and/or bio-burdens in the testing chamber is minimized.

In another embodiment of the present invention, there is provided a method of using the devices disclosed herein to detect an analyte in a specimen, comprising the steps of: adding a specimen into the specimen container; transferring a portion of the specimen from the specimen container to the testing chamber; contacting the specimen with a test element in the testing chamber; and detecting the analyte in the specimen.

### BRIEF DESCRIPTION OF THE DRAWINGS

FIG. 1 is a perspective view of the collecting and test device in one embodiment of the present invention.

FIG. 2A is a top and side perspective view of the container and the test chamber of the collecting and test device in one embodiment of the present invention.

FIG. 2B is a perspective view of the lid of the collecting and test device in one embodiment of the present invention.

FIG. 2C is a bottom and side perspective view of the container and the test chamber of the collecting and test device in one embodiment of the present invention.

FIG. 3A is a bottom and side perspective view of the container and the test chamber containing a test house in a one embodiment of the present invention.

FIG. 3B is a bottom and side perspective view of the container and the test chamber with a test house in one embodiment of the present invention.

FIG. 3C is a perspective view of a test house and a test strip in one embodiment of the collecting and test chamber in the present invention.

FIG. 4A is a cross section side view of the container and the test chamber of the collecting and test chamber in one embodiment of the present invention.

FIG. 4B is a diagrammatic front view of the container of the collecting and test device in one embodiment of the present invention.

FIG. 4C is a cross section top view of the container and the test chamber of the collecting and test device in one embodiment of the present invention.

FIG. 4D is a diagrammatic top view of the container and the test chamber of the collecting and test device in one embodiment of the present invention.

FIG. 5A is a perspective view of the lid of the collecting and test device in one embodiment of the present invention.

FIG. 5B is a front perspective view of the lid of the collecting and test device in one embodiment of the present invention.

FIG. 5C is a cross section side view of the lid of the collecting and test device in one embodiment of the present invention.

FIG. 5D is a diagrammatic top view of the lid of the collecting and test device in one embodiment of the present invention.

FIG. 5E is a diagrammatic bottom view of the lid of the collecting and test device in one embodiment of the present invention.

FIG. 6 is a cross section side view of the collecting and test device with several designs for the conduit, which can be used in the present invention.

FIG. 7A is a perspective view of the collecting and test device in another embodiment of the present invention.

FIG. 7B is an exploded view of the collecting and test device in another embodiment of the present invention.

FIG. 8 is a cross section side view of the test chamber of the collecting and test device in another embodiment of the present invention.

### DRAWING REFERENCE NUMERALS

1. A container in one embodiment of the present invention.
2. The distal end of the container in one embodiment of the present invention.
3. A test chamber in one embodiment of the present invention.
4. The front wall of the test chamber in one embodiment of the present invention.
5. The proximal end of the test chamber in one embodiment of the present invention.
6. The distal end of the test chamber in one embodiment of the present invention.
7. The lid for covering the open proximal end of the container in one embodiment of the present invention.
8. A volume measurement for the container in one embodiment of the present invention.
9. A temperature indicator strip for the container in one embodiment of the present invention.
10. The proximal open end of the container in one embodiment of the present invention.
11. The back wall of the test chamber in one embodiment of the present invention.
12. The threads on the top periphery of the container coupling of the lid to ensure leak-proof in one embodiment of the present invention.
13. A test window of the test chamber in one embodiment of the present invention.
14. The threads along the bottom periphery of the lid coupling of the container to ensure leak proof in one embodiment of the present invention.
15. O-ring structure coupling of the lid of the collecting and test device in one embodiment of the present invention.
16. A conduit for conveying a portion of fluid sample from the container to the test chamber in one embodiment of the present invention.
17. A test strip.
18. A test house in the test chamber of one embodiment of the present invention.
19. The channels of the test chamber.
20. The distal end of the conduit.
21. The opening of the conduit proximal to the distal end of the container in one embodiment of the present invention.
22. The opening of the conduit proximal to a lower end of the back wall of the test chamber in one embodiment of the present invention.
23. A reservoir in the test chamber in one embodiment of the present invention.
24. The proximal open end of the container in another embodiment of the collecting and test device of the present invention.
25. The distal end of the container in another embodiment of the collecting and test device of the present device.
26. A test chamber in another embodiment of the collecting and test device of the present invention.
27. A test window of the test chamber in another embodiment of the collecting and test device of the present invention.
28. The proximal end of the conduit in another embodiment of the collecting and test device of the present invention.
29. The conduit in another embodiment of the collecting and test device of the present invention.
30. The distal end of the conduit in another embodiment of the collecting and test device of the present invention.
31. The opening of the test chamber in another embodiment of the collecting and test device of the present invention.
32. The test result zone of a test strip.
33. The lid for covering the container in another embodiment of the collecting and test device of the present invention.
34. The reagent zone of a test strip.
35. The sample application zone of a test strip.
36. A container in another embodiment of the collecting and test device of the present invention.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down” or “upper” or “lower” and the like refer to orientation of the parts during use of the device. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

An element or structure is “separable from” another element or structure refers an element or structure is capable of being separated, parted or dissociated from another element or structure in the present invention.

An element or structure is “integral to” another element or structure refers the two element or structures are as a single piece or unit in the present invention.

“Proximal” refers to the upper end of an element or structure in the present invention.

“Distal” refers to the end that is opposite to and farthest from the proximal end of the element or structure in the present invention.

“Directly” means that one structure or element is in physical contact with another structure or element, or when used in reference to a procedure, means that one process affects another process or structure without the involvement of an intermediate step or component.

“Indirectly” means that one structure or element is not in immediate physical contact with another structure or element, but rather contacts an intermediary structure or element that contacts the other structure or element. When used in reference to a procedure, “indirectly” means that one process affects another process or structure by means of an intermediate step or component.

A “chemical reagent” refers to any chemical compound, including organic compounds and inorganic compounds and combinations thereof in the present invention. A chemical reagent can be provided in gaseous, solid, or liquid form, or any combination thereof, and can be a component of a solution or a suspension.

A “filter” refers to any substance, such as cloth, paper, porous porcelain, or a layer of charcoal or the like that allows liquid or fluid passing through to remove suspended particles or to recover solids in the present invention.

A “conduit” refers to a pipe, or a tube, or the like for conveying specimen such as liquid or fluid in the present invention.

“Indirect fluid communication” refers to a fluid in one container or structure is not in immediate communication with the fluid in another container or structure, but rather communicates with the fluid in an intermediary container or opening tube structure that communicates with the fluid in another container or structure.

A “test element” refers to an element for analyzing a specimen. A test element can be used to detect the presence and/or concentration of an analyte in a specimen, or to determine the presence and/or numbers of one or more components of a specimen, or to make a qualitative assessment of a sample. Test elements of the present invention include, but are not limited to, enzymatic assay, slides, lateral flow devices such as test strip, test devices, and columns.

A “lateral flow device” is a device that determines the presence and/or amount of an analyte in a liquid specimen as the liquid specimen moves through a matrix or material by lateral flow, such as an immunochromatographic device.

“Analyte” is a compound or composition to be tested in a suspected specimen that is capable of binding specifically to a ligand, receptor, or enzyme, or an antibody or antigen such as a protein or a drug compound, or a drug metabolite. Analytes can include antibodies and receptors, including active fragments or fragments thereof. An analyte can include an analyte analogue, which is a derivative of an analyte, such as, for example, an analyte altered by chemical or biological methods, such as by the action of reactive chemicals, such as adulterants or enzymatic activity.

“Antibody” refers to an immunoglobulin, or derivative or fragment or active fragment thereof, having a binding site that specifically binds to a molecule defined as complementary with a particular spatial and polar organization. The antibody can be monoclonal or polyclonal and can be prepared by techniques that are well known in the art such as, for example, immunization of a host and collection of sera or hybrid cell line technology.

“Specimen” is any material to be tested for the presence and/or concentration of an analyte, or to determine the presence and/or numbers of one or more components in a specimen, or to make a qualitative assessment of a specimen. In one embodiment, a specimen is a fluid, such as a liquid specimen. Examples of liquid specimen that may be tested using a test device of the present invention include, but are not limited to, bodily fluids such as blood, serum, plasma, saliva, urine, ocular fluid, semen, and spinal fluid; water samples such as samples of water from oceans, seas, lakes, rivers, and the like, or samples from home, municipal, or industrial water sources, runoff water or sewage samples; and food samples such as milk or wine. Viscous liquid, semi-solid, or solid specimens may be used to create liquid solutions by extraction, elution or dilution. For example, throat or genital swabs may be suspended in a liquid solution to make a fluid specimen. Specimen can include a combination of liquids, solids, gases, or any combination thereof, as, for example a suspension of cells in a buffer or solution. Specimen can comprise biological materials, such as cells, microbes, organelles, and biochemical complexes. Liquid specimens can be made from solid, semisolid or highly viscous materials, such as soils, fecal matter, tissues, organs, biological fluids or other samples that are not fluid in nature. For example, these solid or semi-solid specimens can be mixed with an appropriate solution, such as a buffer, diluent, extraction buffer, or reagent. The specimens can be macerated, frozen and thawed, or otherwise extracted to form a fluid sample. Residual particulates can be removed or reduced using conventional methods, such as filtration or centrifugation.

Other technical terms used herein have their ordinary meaning in the art that they are used, as exemplified by a variety of technical dictionaries.

The present invention describes a new and improved collecting and testing device, such device comprises a container for collecting and storing a specimen, a test chamber having at least a test element, and a conduit for transferring a specimen from the container to the test chamber. In one embodiment, while the specimen is added into the container, at least a portion of the specimen can automatically enter into the test chamber through the conduit so that the specimen contacts the test element in the test chamber. The conduit allows the fluid transferring from the container to the test chamber. In another embodiment, the specimen in the test chamber is in indirect fluid communication with the specimen in the container, and the specimen stored in the container is separable from the specimen stored in the test chamber, the specimen in the testing chamber cannot flow back into the specimen container, so that the original specimen in the containers remains uncontaminated. The present invention provides such a device and methods of use.

General features of the present invention have thus been broadly outlined in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based may be readily utilized as a basis for designing other structures, methods and systems for carrying out the several purposes of the present invention. It is important, therefore, that the claims be regarded as including such equivalent constructions insofar as they do not depart from the spirit and scope of the present invention.

### Collecting and Testing Device

The present invention describes a new and improved collecting and testing device that includes a container 1 for receiving and/or storing a specimen, a test chamber 3 having at least a test element, and a conduit 16 for transferring a portion of the specimen from the container 1 to the test chamber 3. In one embodiment, while the container 1 receiving the specimen, at least a portion of the specimen can automatically flow into the test chamber 3 through the conduit 16, so that the specimen contacts the test element(s) in the test chamber 3. The conduit 16 allows fluid transferring from the container 1 to the test chamber 3. The specimen in the test chamber 3 is in indirect fluid communication with the specimen in the container 1, and the specimen stored in the container 1 is separable from the specimen in the test chamber 3, the specimen in the testing chamber cannot flow back into the specimen container, so that it minimizes the potential contamination of the original specimen in the container 1 by the chemical regents and/or bio-burdens from a test element of the test chamber 3.

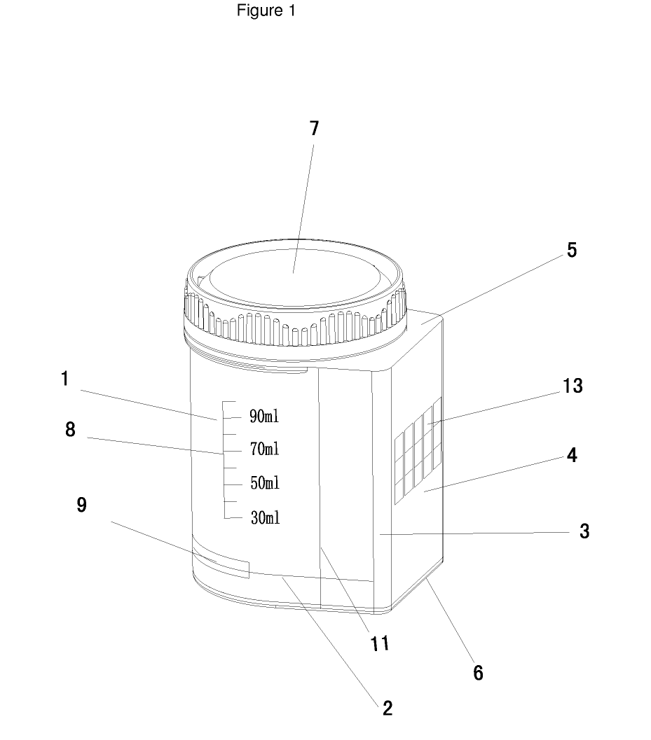

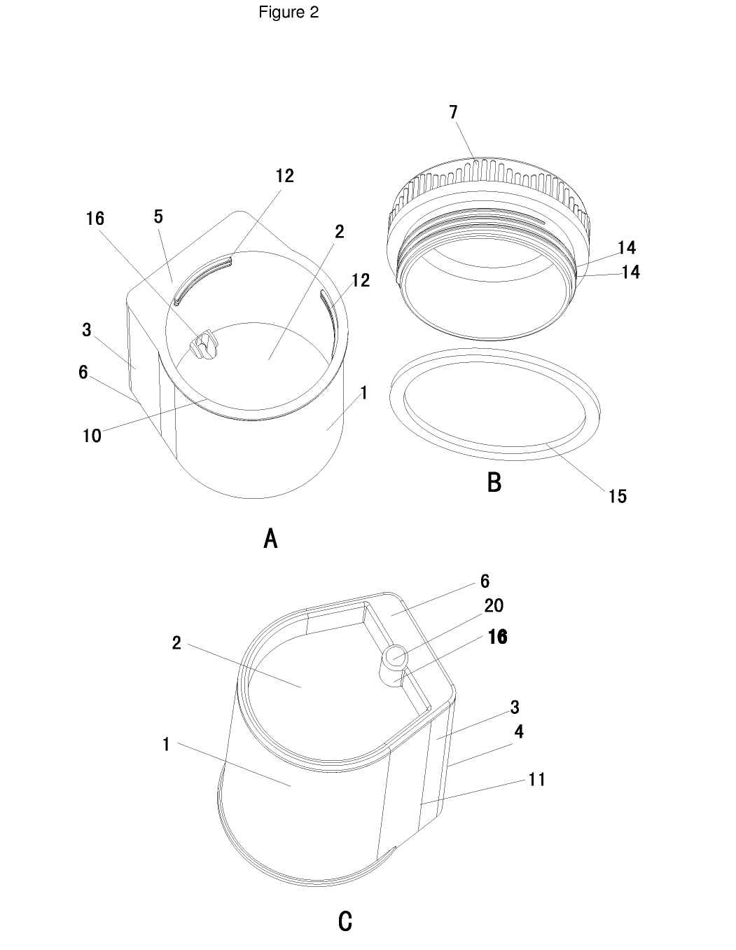

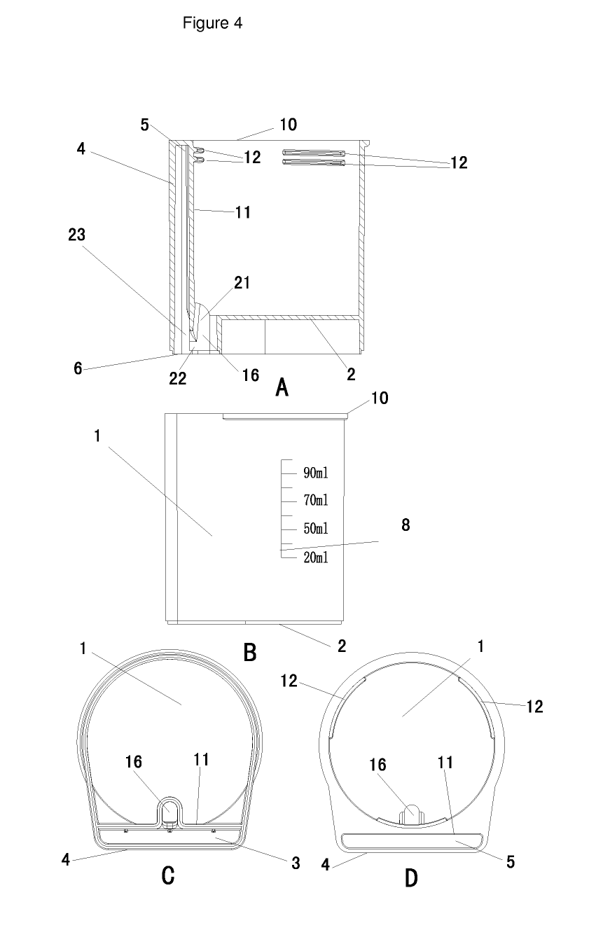

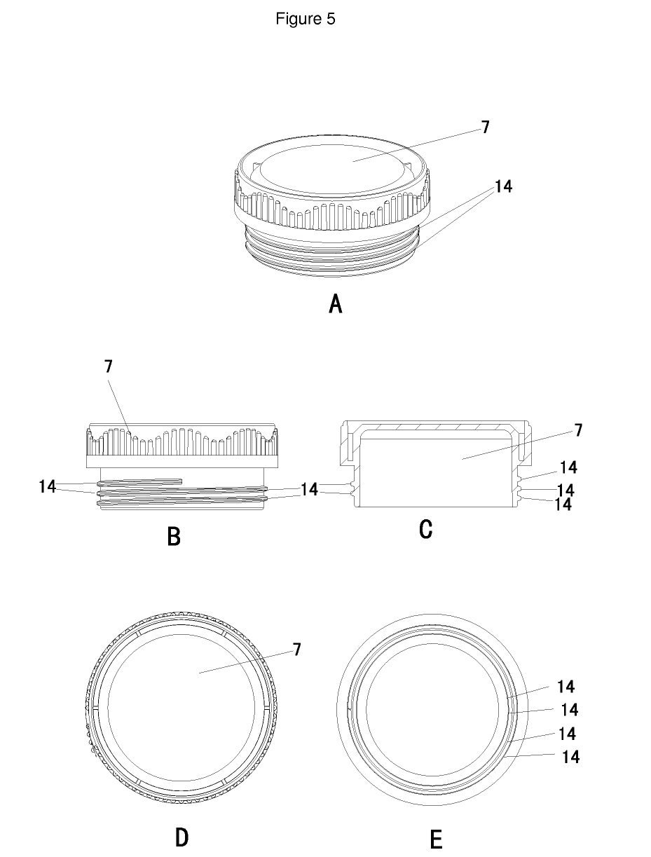

In one embodiment of the present invention, the container 1 and test chamber 3 are substantially parallel as depicted in FIGS. 1, 2, 3, 4 and 6. The container 1 can receive a specimen directly or indirectly by means of a specimen collection device such as, but is not limited to, a swab, rod, spoon, spatula, knife, brush, or fabric. Optionally the container 1 can contain one or more reagents prior to receiving the specimen. In another aspect of the present invention, one or more reagents can be added to the container before, during, or after the container 1 has received the specimen. The specimen can incubate with the reagent or reagents for an approximate or specific period of time prior to adding into the container 1. While the container 1 receiving the specimen with or without one reagent or more reagents, at least a portion of the specimen can automatically release into the test chamber 3 through means of opening tube structures such as, but is not limited to, the conduit 16. Optionally, while the container 1 receiving the specimen, a portion of the specimen can release into the test chamber 3 by penetration of a puncturable barrier in the container 1. Upon releasing from the container 1 into the test chamber 3, the specimen can contact the test element in the test chamber 3. Examples of test elements include, but are not limited to, a lateral flow chromatographic test strip 17.

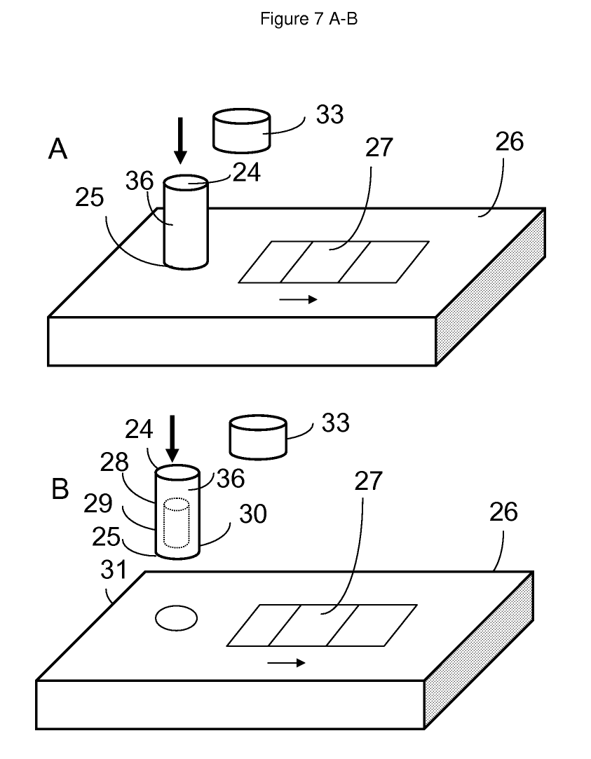

In another embodiment of the present invention, the container 36 and test chamber 26 are substantially perpendicular as depicted in FIG. 7. The container 36 can receive a specimen directly or indirectly by means of a specimen collection device such as, but is not limited to, a swab, rod, spoon, spatula, knife, brush, or fabric. Optionally the container 36 can contain one or more reagents prior to receiving the specimen. In another aspect of the present invention, one or more reagents can be added to the container before, during or after the container 36 has received the specimen. The specimen can incubate with the reagent or reagents for an approximate or specific period of time prior to adding into the container 36. While the container 36 receiving the specimen with or without one or more reagents, at least a portion of the specimen can automatically release into the test chamber 26 through means of opening tube structures such as, but is not limited to, the conduit 29. Optionally, while the container 36 receiving the specimen, a portion of the specimen can release into the test chamber 26 by penetration of a puncturable barrier in the container 36. Upon release from the container 36, the specimen, can contact with the test element in the test chamber 26. Examples of test elements include, but are not limited to, a lateral flow chromatographic test strip.

### Specimen Container

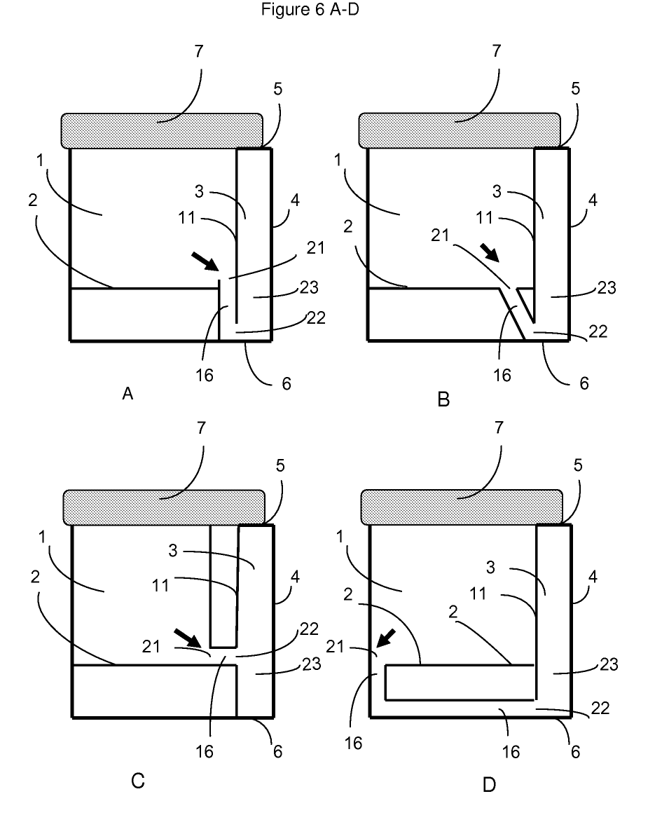

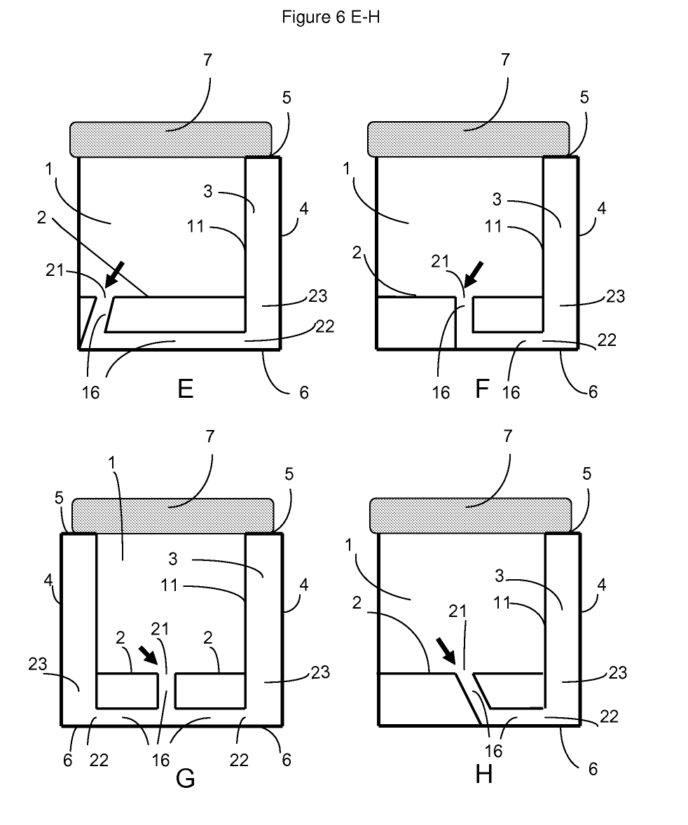

In one embodiment of the present invention, the container 1 is substantially parallel to the test chamber 3 as depicted in FIGS. 1, 2, 3, 4 and 6. The specimen container 1 comprises an open proximal end 10 and a distal end 2. In one embodiment, the container 1 receives a specimen through the open proximal end 10, and the distal end 2 has an opening 21 connecting the conduit 16 for conveying a portion of specimen into the test chamber 3. In one embodiment of the present invention, the distal end 6 of the test chamber 3 is in a lower position than that of the distal end 2 of the container 1, so that a portion of the specimen in the container 1 can readily flow into the test chamber 3 through the conduit 16 by gravitational force as depicted in FIGS. 1, 2, 3, 4 and 6. In another embodiment, an opening 21 of the conduit 16 can be positioned at the low portion of the cylindrical wall of container 1 as depicted in FIG. 6C. The specimen container 1 can be of any geometric shape or dimension such as, but not limited to, triangular, spherical, oval, square, rectangular, pentagonal, hexagonal, heptagonal, octagonal, or any polygon, or non-geometric shape such as kidney or bean shaped. In one embodiment, the specimen container is substantially cylindrical. The size of the specimen container 1, encompassing such dimensions as the width, height and diameter of the specimen container can be such that an indiscriminate or predetermined volume of a specimen can be efficiently transferred into the container 1, or can readily accept insertion of a specimen collection device and if desirable, one or more reagents. The proximal end 10 of the container 1 can be circle, flared, funnel shaped or otherwise molded such that a specimen can readily and/or accurately be added into the specimen container 1. Alternatively, an adaptor can be separable and directly or indirectly engage the proximal end 10 of the container 1.

The container 1 can be made of suitable material such as, but not limited to, glass, ceramics, metals, plastics, polymers, or copolymers, or any combination thereof. In one embodiment, the material comprises a plastic, polymer or copolymer such as those that are resistant to breakage, such as polypropylene, polyallomer, polycarbonate or cycloolefins or cycloolefin copolymers. The container 1 can be made by appropriate manufacturing methods, such as, but not limited to, injection molding, blow molding, machining or press molding.

A specimen can be fluid, solid or gaseous, or any combination thereof. In one aspect of the present invention, a specimen can be transferred to, or added to, or flow to the container 1. Transfer of a specimen into the container 1 can be by various techniques such as, but not limited to pipetting, pouring, decanting, dropping, or streaming. In one embodiment, a specimen can be mixed with one or more reagents. For example, a specimen mixing with one or more reagents can occur prior to transferring into the container 1. Reagents can include one or more chemical components, such as, but not limited to, salt, chelators, anticoagulants, detergents, stabilizers, diluents, buffer, enzymes, cofactors, specific binding factors, and the like. The one or more reagents can be mixed to facilitate analysis of a specimen, but this is not a requirement of the present invention.

In one embodiment of the present invention, a specimen can be transferred to the container 1 by means of a specimen collection device such as, but not limited to, a swab, rod, spoon, spatula, knife, brush, or fabric. The specimen collection device with specimen can be transferred or otherwise placed or inserted into the container 1. Optionally one or more reagents have been added into the container 1 or subsequently added to the container 1.

In one embodiment of the present invention, an opening 21 of the conduit 16 is placed at or near the distal end 2 of the specimen container 1, and another opening 22 of the conduit 16 is placed at or near the distal end of the back wall 11 of the test chamber 3, further the level of the distal end 2 of the container 1 is in a higher position than that of the distal end 6 of the test chamber 3 depicted as FIGS. 1, 2, 3, 4 and 6, so that a portion of the specimen can readily flow into the test chamber 3 from the container 1 through the conduit 16 by gravitational force. In another embodiment of the present invention, the length of the conduit 16 can be substantially parallel to the distal end 2 of the container 1, or be formed at an appropriate angle with the distal end 2 of the container 1, or substantially perpendicular to the distal end 2 of the container 1 as depicted in FIGS. 6A, 6B, 6C, 6D, 6E, 6F, 6G and 6H. The conduit 16 can be configured into any shape such as, but not limited to triangular, spherical, oval, square, rectangular, pentagonal, hexagonal, heptagonal, octagonal, or any polygon, or non-geometric shape such as kidney or bean shaped. In one embodiment, the conduit is substantially cylindrical. The size of the conduit 16, encompassing such dimension as the length, width, and diameter controls the volume of the specimen entering into the test chamber 3. Generally the size of conduit 16 allows a desirable amount of specimen enters into the test chamber 3 from the container 1 so that the specimen in the test chamber 3 can efficiently contact the test element in the test chamber 3, but not overload the reservoir 23 of the test chamber 3. In one embodiment, the conduit 16 allows transfer of about 0.1 to 10 milliliter of the specimen from the container 1 to the test chamber 3. Furthermore, the specimen in test chamber 3 is in indirect fluid communication with the specimen in container 1, and the specimen stored in container 1 is separable from the specimen in test chamber 3, the specimen in the testing chamber cannot flow back into the specimen container so that the original specimen in container 1 remains uncontaminated by the chemical regents and/or bio-burdens from a test element of the test chamber 3.

In another embodiment of the present invention, the container 36 and test chamber 26 are substantially perpendicular as depicted in FIG. 7. The specimen container 36 comprises an open proximal end 24 and a distal end 25, wherein the open proximal end 24 can receive a specimen and the distal end 25 is connected to conduit 29 for transferring a portion of the specimen into the test chamber 26. In one embodiment, while a specimen is being added into container 36, at least a portion of the specimen in container 36 can automatically enter into the test chamber 26 through conduit 29 as depicted in FIG. 7. The specimen container 36 can be of any geometric shape or dimension such as, but not limited to, triangular, spherical, oval, square, rectangular, pentagonal, hexagonal, heptagonal, octagonal, or any polygon, or non-geometric shape such as kidney or bean shaped. In one embodiment, the specimen container is substantially cylindrical. The size of the specimen container 36, encompassing such dimensions as the width, height and diameter of the specimen container can be such that an indiscriminate or predetermined volume of a specimen can be efficiently transferred to the container 36, or can readily accept insertion of a specimen collection device, and if desirable, one or more reagents. The proximal end 24 of the container 36 can be circle, flared, funnel shaped or otherwise molded such that a specimen can readily and accurately be added into the specimen container 36. Alternatively, an adaptor can separable and directly or indirectly engage the proximal end 24 of the container 36.

Container 36 can be made of suitable material such as, but not limited to, glass, ceramics, metals, plastics, polymers, or copolymers, or any combination thereof. In one embodiment, the material comprises a plastic, polymer or copolymer such as those that are resistant to breakage, such as polypropylene, polyallomer, polycarbonate or cycloolefins or cycloolefin copolymers. Container 36 can be made by appropriate manufacturing methods, such as, but not limited to, injection molding, blow molding, machining or press molding.

A specimen can be fluid, solid or gaseous, or any combination thereof. Transfer of a specimen into the container 36 can be by various techniques such as, but not limited to, pipetting, pouring, decanting, dropping or streaming. Optionally, a specimen can be mixed with one or more reagents. For example, mixing can occur prior to transfer into the container 36. Reagents can include one or more chemical components, chelators, anticoagulants, detergents, stabilizers, diluents, buffer, enzymes, cofactors, specific binding factors, and the like. In one embodiment, the one or more reagents are added to facilitate analysis of a specimen.

In another aspect of the present invention, a specimen can be transferred to the container 36 by means of a specimen collection device such as, but not limited to, a swab, rod, spoon, spatula, knife, brush, or fabric. The specimen collection device with specimen can then be transferred or otherwise placed or inserted into the container 36, optionally with one or more reagents in the container 36 or subsequently added to the container 36.

Conduit 29 can be located at the inside of the container 36, or outside of the container 36, or partially inside and partially outside of the container 36. In one embodiment, the conduit is placed inside the container 36 (for example see FIG. 7). A proximal opening end 28 of the conduit 29 can be positioned at or near the proximal end 24 of the specimen container 36. A distal opening end 30 of the conduit 29 can be placed at or near the proximal or distal end of the test chamber 26, so that a portion of the specimen from container 36 can readily enter into the test chamber 26 through conduit 29 by gravity. The length of the conduit 29 can be substantially parallel to the distal end 25 of the container 36, or form an appropriate angle with the distal end 25 of the container 36. In one embodiment, the conduit 29 is substantially perpendicular to the distal end 25 of container 36. The conduit 29 can be any shape such as, but not limited to, triangular, spherical, oval, square, rectangular, pentagonal, hexagonal, heptagonal, octagonal, or any polygon, or non-geometric shape such as kidney or bean shaped. In one embodiment, the conduit 29 is substantially cylindrical. The size of the conduit 29, encompassing such dimension as the length, width, and diameter controls the volume of the specimen entering into the test chamber 26. Generally the size of the conduit allows a desirable amount of specimen enters into the test chamber 26 from container 36 so that the specimen in the test chamber can efficiently contact the test element in test chamber 26, but not overload the test chamber 26. In one embodiment, such conduit allows transfer of about 0.1 to 10 milliliter of the specimen to the test chamber 26. Preferably the specimen in test chamber 26 does not directly contact the original specimen in the container 36, or separates or is separable from the specimen in the container 36 so that the specimen in the original container remains uncontaminated by the chemical regents and/or bio-burdens from a test element of the test chamber 36.

In another embodiment of the present invention, the distal end 2 of container 1 can include a filter. In one embodiment, the filter can be placed inside of the conduit 16. The filter may be substantially water permeable on the side facing the container 1, but is substantially water impermeable on the side facing the test chamber 3 so that a portion of fluid in container 1 is allowed to flow into the test chamber 3, but the fluid in test chamber 3 is not allowed flowing back into the container.

In another embodiment, a membrane material can be included at or near the distal end 2 of the container 1. The membrane material can be dissolved over time after coming into contact with a specimen or specimen and reagent. Such a membrane can be formed of a material such as, but not limited to, polysaccharides, starches, gelatins, plastics, or the like, or any combination thereof. In another aspect of the present invention, a predetermined amount of one or more reagents can be prepackaged in the container 1. The reagents can be dissolved after coming into contact with a specimen or specimen and reagent. Reagents can include one or more chemical components such as salts, chelators, anticoagulants, detergents, stabilizers, diluents, buffer, enzymes, cofactors, specific binding factors, and the like.

In another embodiment of the present invention, the distal end 25 of container 36 can include a filter. In one embodiment, the filter can be placed inside of the conduit 29. For example, such filter is substantially water permeable on the side facing the container 36, but is substantially water impermeable on the other side facing the test chamber 26 so that a portion of fluid in the container 36 is allowed to flow into the test chamber 26, but the fluid in test chamber 26 is not allowed flowing back to container 36.

In another embodiment, a membrane material can be included at or near the distal end 25 of container 36. The membrane material can be dissolved over time after coming into contact with a specimen or specimen and reagent. Such a membrane can be formed of a material such as, but not limited to, polysaccharides, starches, gelatins, plastics, or the like, or any combination thereof. In another embodiment, a predetermined amount of one or more reagents can be prepackaged in container 36. The reagents can be dissolved after coming into contact with a specimen or specimen and reagent. Reagents can include one or more chemical components such as salts, chelators, anticoagulants, detergents, stabilizers, diluents, buffer, enzymes, cofactors, specific binding factors, and the like.

### Test Chamber

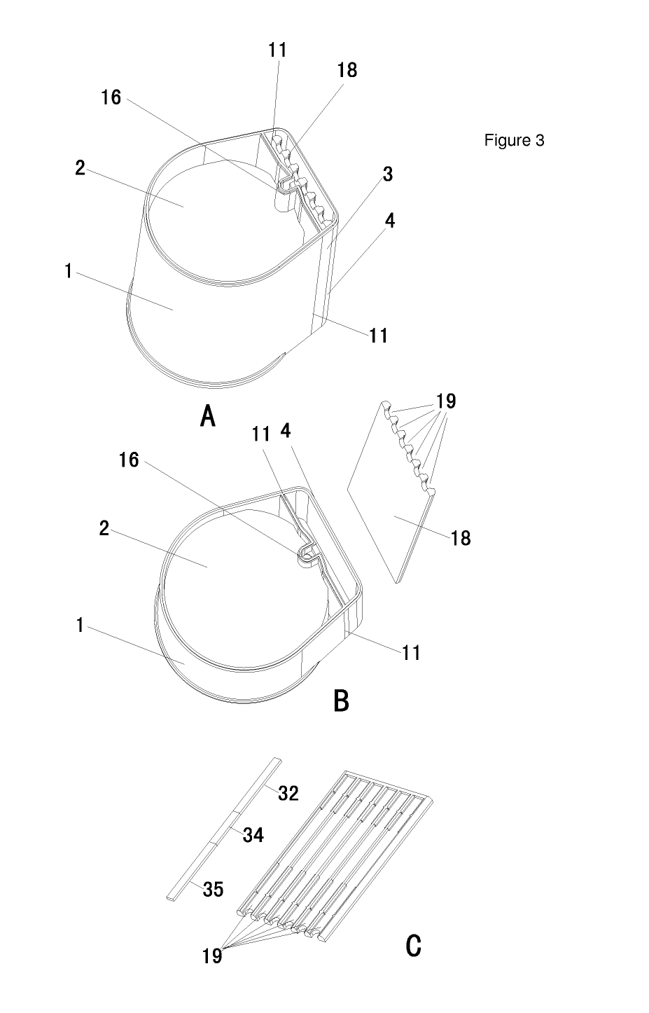

In one embodiment of the present invention, the test chamber 3 of the collecting and testing device comprises one or more housings for holding one or more test elements such as, but not limited to, a lateral flow test device such as a test strip as depicted in FIG. 3C. In one embodiment, the longitudinal section of the test chamber 3 is substantially parallel to the container 1 (for example see FIGS. 1, 2, 3, 4 and 6). The test chamber 3 contains one or more openings 22 near or at the distal end of the back wall 11 of the test chamber 3 for receiving specimen from the container 1 through the conduit 16. The conduit 16 functions as an intermediary structure involving fluid communication from the container 1 to the test chamber 3 so that the container 1 and the test chamber 3 is in indirect fluid communication. The sample application area of at least one test element is positioned at or near the opening 22 of the test chamber 3 so that the fluid contents from the container 1 through the conduit 16 can readily come into contact with the test element in the test chamber 3. Alternatively, one or more absorbent materials such as fiber glass, absorbent paper can be placed at or near the opening 21, and/or the opening 22 of the conduit 16 for facilitating fluid communication with the test element in the test chamber.

In one embodiment of the present invention, the test chamber comprises a front wall 4, a back wall 11, a proximal end 5 and a distal end 6 as depicted as FIGS. 1, 2, 3, 4 and 6, so that air or gas cannot access the test chamber except through an opening 22. The test chamber 3 is capable of trapping a limited amount of air or gas inside of the test chamber 3 and creating a downward pressure to limit the amount of the specimen entering into the test chamber 3 through the opening 22. Thus the test chamber 3 or the reservoir 23 in the test chamber 3 is not overloaded with the specimen. In one embodiment of the present invention, the distal end 6 of the test chamber 3 is in a lower position than that of the distal end 2 of the container 1, so that a portion of the specimen in the container 1 can readily flow into the test chamber 3 through the conduit 16 by gravitational force as depicted in FIGS. 1, 2, 3, 4 and 6. Furthermore, the specimen trapped in the test chamber 3 is incapable of flowing back to the container 1. Optionally, a seal can be included in the proximal end 5 of the test chamber 3. The seal can be, but not limited to, a plug, film, cap, or lid, and can be made by self-adhesive paper, wax paper, plastic material, thin metal film, rubber material, or any other suitable materials. The seal can be removable or not removable after insertion into the proximal end of the test chamber.

The test chamber 3 of the collecting and testing device in the present invention can be made of, but not be limited to, any suitable material, such as glass, ceramics, metals, paper, pressed cardboard, or polymers. In one embodiment, the test chamber is made of plastic, polymer or copolymer such as those that are resistant to breakage, such as polypropylene, polyallomer, polycarbonate or cycloolefins or cycloolefin copolymers. The test chamber 3 can be of any shape or depth such as triangular, spherical, oval, square, rectangular, pentagonal, hexagonal, heptagonal, octagonal, or any polygon, or non-geometric shape such as kidney or bean shaped. In one embodiment, the test chamber is substantially rectangular. The test chamber 3 may be integral to the container 1 to form a single unit. Alternatively, the test chamber 3 and the container 1 form separate units, the test chamber 3 is attachable with and is attached to the container 1 by suitable means of attachment including, but not limited to, thermal welding, ultrasonic welding, snap coupling, gluing, screw, nails, sliding, etc.

One or more test elements, e.g. one or more test strips, can be housed in the test chamber 3. In one embodiment, the test chamber 3 has one or more test houses that comprise one or more channels that are substantially along the test chamber 3. The dimensions of such channels can accommodate a test element, e.g. a test strip 17. The one or more channels can be open, that is uncovered, or one or more windows 13 can be positioned to cover the one or more channels and test elements such that flow and visual results can be observed in accordance with the test and the test element. A window 13 can consist of any transparent material, such as glass, plastic, or mylar, but is preferably break resistant materials. In general, window 13 is moisture resistant such that the one or more test elements are shielded from external moisture.

In one embodiment, the test chamber 3 can have one or more openings 22 at or near the distal end of the back wall 11 so that the test chamber can receive a specimen or specimen and one or more reagents from the container 1. For example, at least one or more openings 22 can be positioned at or near the end of at least one channel 19. In one embodiment, the one or more openings 22 can be at or near the end of the one or more channels 19 such that a sample application zone 35 of one or more test elements (e.g. a test strip) is accessible to fluid communication with a specimen or specimen and one or more reagents. The one or more channels 19 can be open, that is uncovered, or one or more windows can be positioned to cover the one or more channels and test elements such that flow and visual results can be observed in accordance with the test and the test element.

In another embodiment, the test chamber 3 has one or more openings 22 that lead to a common sample application zone of a test element (e.g. a test strip). The test strips 17 can be aligned in parallel (for example see FIG. 3) or be juxtaposed to each other in any pattern. Alternatively a single opening can be associated with a plurality of test strips. For example, a specimen or specimen and reagent can be made available through a single opening 22 to each of a plurality of test strips such that the specimen can come into fluid communication with the test strips that can test for the presence or absence of different analytes. The plurality of test strips can radiate from the single opening in all directions or in a confined array, or any combination thereof. A test chamber can have one or more openings that can give access to the sample application zone of one or more test strips.

In another embodiment, the test chamber 26 is substantially perpendicular to the container 36 as depicted in FIG. 7. The test chamber 36 comprises an opening 31 near or at the proximal end of the test chamber 26 for receiving the specimen from the container 36 of the collecting and testing device. The conduit 29 functions as an intermediary structure involving fluid communication from the container 36 to the test chamber 26, therefore container 36 and test chamber 26 are in indirect fluid communication. The sample application zone of at least one test element is positioned at or near the opening 31 of the test chamber 26 such that the fluid contents from the container 36 through the conduit 29 can readily come into contact with the test element. Alternatively, one or more absorbent materials such as fiber glass paper or absorbent paper can be placed at or near the opening 31 for facilitating fluid in contact with the test element in the test chamber.

The test chamber 26 can be made of, but not be limited to, any suitable material, such as glass, ceramics, metals, paper, pressed cardboard, or polymers. In one embodiment, the material comprises a plastic, polymer or copolymer such as those that are resistant to breakage, such as polypropylene, polyallomer, polycarbonate or cycloolefins or cycloolefin copolymers. The test chamber 26 can be of any shape or depth such as triangular, spherical, oval, square, rectangular, pentagonal, hexagonal, heptagonal, octagonal, or any polygon, or non-geometric shape such as kidney or bean shaped. In one embodiment, the test chamber is substantially rectangular.

One or more test elements, e.g. one or more test strips, can be housed in the test chamber 26. In one embodiment, the test chamber has one or more test houses that comprise one or more channels that are substantially along the test chamber. The dimensions of such channels can accommodate a test element, e.g. a test strip. The one or more channels can be open, that is uncovered, or one or more windows can be positioned to cover the one or more channels and test elements such that flow and visual results can be observed in accordance with the test and the test element. A window 27 can consist of any transparent material, such as glass, plastic, or mylar, or preferably break resistant materials. In general, the window 27 covering at least one channel of the test chamber 26 is moisture resistant so that the one or more test elements are shielded from external moisture.

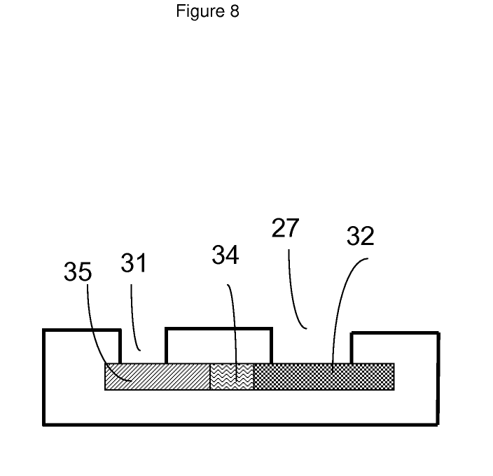

In one embodiment, the test chamber 26 can have one or more openings 31 that can receive a specimen or specimen and one or more reagents into the test chamber 26. For example, the at least one or more openings 31 are positioned at or near the end of at least one channel of the test chamber having at least one test element. In one embodiment, the one or more openings 31 can be at or near the end of the one or more channels such that a sample application zone 35 of one or more test elements, e.g. a test strip, is accessible to fluid communication with a specimen or specimen and one or more reagents (for example see FIG. 8). The one or more channels can be open, that is uncovered, or one or more windows can be positioned to cover the one or more channels and test elements such that flow and visual results can be observed in accordance with the test and the test element.

In another embodiment, test chamber 26 can has one or more openings leading to a common sample application zone 35 of one or more test elements such as a test strip. The test strips can be aligned in parallel or be juxtaposed to each other in any pattern. Alternatively, a single opening can be associated with a plurality of test strips. For example, a specimen or specimen and reagent can be made available through a single opening to each of a plurality of test strips such that the specimen or specimen and reagent can come into fluid communication with the test strips that can test for the presence or absence of different analytes. The plurality of test strips can radiate from the single opening in all directions or in a confined array, or any combination thereof.

### Test Element

The test element housed within the test chamber of the collecting and testing device of the present invention can be any test element known in the art. In one embodiment, the test element comprises a lateral flow test device such as a test strip, e.g. an immunological test strip (For examples see FIGS. 3C and 8). The test chamber of the present invention can house one or more test elements such as test strips. The one or more test strips can be of any shape and dimensions, e.g. it is a rectangular test strip (For example, see FIGS. 3C and 8).

In one embodiment of the present invention, the test strip may comprise, at least in part, any bibulous material, such as, but not limited to nylon, paper, glass fiber, dacron, polyester, nitrocellulose, polyethylene, olefin, or other thermoplastic materials such as polyvinyl chloride, polyvinyl acetate, copolymers of vinyl acetate and vinyl chloride, polyamide, polycarbonate, polystyrene, etc. In one embodiment, at least one test strip comprises the material such as nitrocellulose having a pore size of at least about 1 micron, more preferably of greater than about 5 microns, or about 8-12 microns. Suitable nitrocellulose sheets having a nominal pore size of up to approximately 12 microns are available commercially from, for example, Schleicher and Schuell GmbH.

The test strips are preferably in fluid communication as depicted in FIGS. 3C and 8. One material of a test strip may be overlaid on another material of the test strip, such as for example, a filter paper is overlaid on nitrocellulose. Alternatively, a test strip may include a region comprising one or more materials followed by a region comprising one or more different materials. In this case, the regions are in fluid communication and may or may not partially overlap one another.

In one embodiment of the present invention, the test strips include a sample application zone 35 and a test result zone 32. The test result zone 32 can include either or both of one of more analyte detection zones and one or more control zones. Optionally, a test strip can include a reagent zone 34.

One or more reagents for binding one or more analyte in the specimen can be impregnated throughout the thickness of the material of the test result zone 32 (for example, the reagents for binding one or more analytes, as well as the reagents for binding one or more control analytes, can be impregnated throughout the thickness of the test result zone). Such impregnation can enhance the extent to which the immobilized reagent can capture an analyte or more analytes present in the migrating specimen. Alternatively, the reagents, including those for binding one or more analytes, or one or more control analytes, may be applied to the surface of the bibulous or non-bibulous material in the test result zone 32. Impregnation of reagents into the test strip materials or application of specific reagents onto test strip materials may be done manually or by machine.

Nitrocellulose has the advantage that one or more reagents can be immobilized in the test result zone 32 without prior chemical treatment. If a porous solid phase material comprises paper, for example, immobilization of a reagent such as antibody in the test result zone can be performed by chemical coupling using, for example, CNBr, carbonyldiimidazole, or tresyl chloride.

Following the impregnation of one or more reagents into the test result zone 32, the remainder of the porous solid phase material should be treated to block any remaining binding sites. Blocking can be achieved by treatment with protein (for example bovine serum albumin or milk protein), or with polyvinylalcohol or ethanolamine, or any combination of these agents. A labeled reagent for the reagent zone 34 can then be dispensed onto the dry solid phase material and will become mobile in the carrier when in the wet state. Between each of these various process steps the porous solid phase material should be dried.

To help the movement of a labeled reagent throughout the test strip when the test strip is moistened with specimen, a labeled reagent can be dispensed to the bibulous or non-bibulous material in a surface layer, or throughout the thickness of the bibulous or non-bibulous material. To minimize interaction between the bibulous or non-bibulous material and the labeled reagent, a glazing material can be mixed with a labeled reagent prior to dispensing to the bibulous or non-bibulous material, or impregnated in the region to which the labeled reagent is to be applied. Glazing can be achieved, for example, by depositing the mixture of an aqueous sugar or cellulose solution, for example of sucrose or lactose with the labeled reagent, on the test element at the relevant portion, and then drying (see, U.S. Pat. No. 5,656,503). Alternatively, the labeled reagent can be applied to the glazed portion of the test element. The remainder of the test element material cannot be glazed.

The reagents can be applied to the test element material in a variety of ways. Various “printing” techniques have previously been proposed for application of liquid reagents to carriers, including but not limited to micro-syringes, pens using metered pumps, direct printing and ink-jet printing, and any of these techniques can be used in the present context.

### Sample Application Zone 35

The sample application zone 35 is an area of a test element/strip where a specimen, such as a fluid sample, including but not limited to, a biological fluid sample such as blood, serum, saliva, urine, or a fluid derived from a biological sample, such as a throat or genital swab, is applied. The sample application zone 35 may comprise a bibulous or non-bibulous material, such as but not limited to filter paper, nitrocellulose, glass fibers, polyester or other appropriate materials. One or more materials of the sample application zone may perform a filtering function, such that large particles or cells are prevented from moving through the test strip.

The sample application zone 35 can be in direct or indirect fluid communication with the rest of the test strip, including the test result zone 32. Such direct or indirect fluid communication includes, but not limited to, end-to-end communication, overlap communication, or overlap or end-to-end communication that involves another element, such as a fluid communication structure such as filter paper. The sample application zone 35 may also comprised one or more compounds or molecules that may be necessary or desirable for optimal performance of the test. Examples of such compounds include, but are not limited to, buffers, stabilizers, surfactants, salts, reducing agents, or enzymes.

### Reagent Zone 34

The test strip may also comprise a reagent zone 34 where reagent for the detection of an analyte may be immobilized (e.g. via covalent or non-covalent immobilization) or not immobilized, particularly when in a wet state. The reagent zone 34 may be on a reagent pad, a separate segment of bibulous or non-bibulous material of the test strip, or it can be part of the region on a test strip such as the test result zone 32. In one aspect of the invention, the reagent zone 34 comprises a labeled reagent, such as antibodies or active fragments thereof attached or linked to a label. Such labeled reagent can be made using methods known in the art. The specific reagent can bind an analyte and/or a control compound.

In another aspect of the invention, the reagent zone 34 includes an analyte or an analyte analog bound to a population of labeled reagent. The analyte in the sample competes with the analyte or analyte analog in the reagent zone 34 for binding to a labeled reagent. A reduced visual signal in comparison with a control sample lacking analyte indicates the presence of analyte in the sample. In another embodiment of the invention, the reagent zone 34 may include one or more components of a signal producing system, for example, catalysts, such as enzymes, cofactors, electron donors or acceptors, and/or indicator compounds. The reagent zone 34 may also include compounds or molecules that may be necessary or desirable for optimal performance of the test, for example, buffers, stabilizers, surfactants, salts, reducing agents, or enzymes.

### Test Result Zone 32

The test result zone 32 comprises one or more immobilized or not immobilized reagents that can detect the presence of an analyte in the specimen, such as but not limited to, drugs of abuse, hormones, metabolites, and antibodies. Such reagents are preferably in a dry state and can be covalently immobilized, non-covalently immobilized, or not immobilized in the test result zone 32. The test result zone 32 can also include a control zone. The control zone can be upstream from, downstream from, or integral with the analyte detection zone of the test result zone 32. The control zone provides a result that indicates the test on the test strip has performed correctly.

In another aspect of the present invention, a test strip can optionally include an adulteration control zone that is capable of detecting an adulteration analyte or an adulteration indicator. Such an adulteration control zone can be in addition to or in place of a control zone or a test result zone of the test strip as described herein.

### A Method of Detecting an Analyte in a Specimen

The methods disclosed herein may be performed by using a variety of test device configurations as previously described and as set forth in the disclosed methods and examples. The collecting and testing device of the present invention can be used to collect a specimen to a container 1 or 36 and optionally mix the specimen with one or more reagents. A portion of the specimen or specimen and one or more reagents can then automatically enter a test chamber 3 or 26 comprising at least a test element to detect one or more analytes in the specimen. The conduit 16 or 29 is an intermediary structure for fluid communication from the container 1 or 36 to the test chamber 3 or 26, therefore the specimen in the container 1 or 36 is in indirect fluid communication with the specimen in the test chamber 3 or 26. Furthermore, the specimen stored in the container 1 or 36 is separable from the specimen in the test chamber 3 or 26, so that the original specimen in the containers 1 or 36 remains uncontaminated. The specimen can be gaseous, liquid, colloidal or solid, or any combination thereof, e.g. a biological fluid specimen. Examples of a biological fluid specimen include, but are not limited to, blood, serum, saliva, or urine. Other biological specimens include fecal samples, and throat or genital swabs. Examples of solid specimens include such materials as dirt, grains, granules, powders or pellets. In one aspect of the present invention, a specimen can be transferred to, or flow into container 1 or 36. Transfer of a specimen into container 1 or 36 can be by various techniques such as, but not limited to, pipetting, pouring, decanting, dropping or streaming. Optionally, a specimen can be mixed with one or more reagents. For example, mixing can occur prior to transfer into container 1 or 36. Reagents can include one or more chemical components, chelators, anticoagulants, detergents, stabilizers, diluents, buffer, enzymes, cofactors, specific binding factors, and the like. The one or more reagents can be mixed to facilitate analysis of a specimen. In another aspect of the present invention, a specimen can be added to the container 1 or 36 by means of a specimen collection device such as, but not limited to, a swab, rod, spoon, spatula, knife, brush, or fabric. The specimen collection device together with the specimen can then be transferred or otherwise placed or inserted into container 1 or 36. One or more reagents may be added prior or subsequently to the container 1 or 36.

The container 1 or 36 can be integral to the test chamber 3 or 26, or can be separable from the test chamber 3 or 26. In each instance the conduit 16 or 29 is an intermediary structure for fluid communication from the container 1 or 36 to the test chamber 3 or 26. The specimen in the container 1 or 36 is in indirect fluid communication with the specimen in the test chamber 3 or 26. Furthermore, the specimen stored in the container 1 or 36 is separable from the specimen stored in the test chamber 3 or 26.

The test chamber 3 or 26 of the present invention can house at least a test element, e.g. an immunological test strip 17. The collecting and testing device of the present invention can be used to determine whether a specific analyte is present in a specimen. The analyte of interest can be of various kinds, for example a biological moiety such as an antibody or antigen or a hormone such as hCG (human chorionicgonadotropin); a drug or chemical moiety; or an etiological agent or extract from an etiological agent such as Streptococcus or HIV (human immunodeficiency virus). Sample application zone 35 of one or more test strips can be positioned immediately below or in the vicinity of an opening of the test chamber. The specimen or specimen and reagent travels by capillary flow along the immunochromatographic test strip and the presence or absence of an analyte in the specimen can be determined by the presence or absence of a visual line in the test result zone 32 of a test strip as viewed through an opening or window 17 or 27 on the test chamber 3 or 26.

The invention being generally described, will be more readily understood by reference to the following examples which are included merely for purposes of illustration of certain aspects and embodiments of the present invention, and are not intended to limit the invention.

### EXAMPLE 1

A collecting and testing device depicted as FIGS. 1, 2, 3 and 4 containing one or more test strips can be used for the detection of drug(s) or drug metabolites in human urine. A subject in need for a drug test is given the collecting and testing device or referred as “the cup”. In the restroom, the subject opens the lid of the cup and urinates into the container of the cup, then closes the cup and gives the cup to a technician. When the person urinates into the container of the cup, a portion of the urine automatically flows into the test chamber through the conduit. The urine in the test chamber contacts the sample application zone of one or more test strips, and the testing is initiated. The test result can be viewed through the window of the test chamber.

The technician reports the results in Example 1. If the result is a preliminary positive, the cup with urine specimen will be sent to a Lab for confirmatory test. The urine specimen stored in the container can be separable from the urine specimen in the test chamber, and the specimen trapped in the test chamber of the cup is incapable of flowing back to the container of the cup, so that the urine specimen stored in the container of the cup remains uncontaminated by chemical reagents from the test strips in the test chamber.

### EXAMPLE 2

The collecting and testing device depicted in FIGS. 7 and 8 containing at least a test strip can be used for the detection of Strep-A. A throat specimen is obtained from a patient exhibiting signs and symptoms of pharyngitis using a standard size rayon or dacron swab. Four to five drops or approximately 200 microliters of reagent A (2 molar sodium nitrate) and four to five drops, approximately 200 microliters of reagent B (0.2 molar acetic acid) are added to a collection device such as a test tube. The swab containing the throat specimen is inserted into the tube and then allowed to incubate for 60 seconds. Then, the liquid contents in the tube are transferred to the specimen container of the present invention, wherein a portion of the liquid contents (approximately 150 microliter) will flows into the test chamber through the conduit. A liquid sample flow is initiated on the test strip by capillary action and the result of the test can be viewed through the test result window in about 5 minutes. The rest of the liquid sample in the container can be used for a confirmatory test if needed.

### EXAMPLE 3

The collecting and testing device depicted in FIGS. 7 and 8 containing at least a test strip can be used for the detection of Chlamydia. Endocervical specimen is collected using either rayon or dacron swabs with plastic shafts or a cytobrush. One hundred and sixty (160) microliters of 1 normal potassium hydroxide is placed into a collection device such as a test tube. The swab or brush is placed into the tube, mixed for 10-20 seconds, and allowed to incubate for 5 minutes. After this time, 160 microliters of 1 molar acetic acid containing 0.1% of Tween-20 are added to the tube. The swab or brush is rotated for an additional 10-20 seconds. The liquid contents in the tube, approximately 300 microliters, are then added to the specimen container of the present invention. A portion of the liquid contents (approximately 150 microliters) will automatically enter into the test chamber through a conduit. Sample flow is initiated on the test strip by capillary action and the result of the test can be viewed through the test result window in about 10 minutes. The rest of the liquid sample in the container can be used for a confirmatory test if need

### EXAMPLE 4

The collecting and testing device depicted in FIGS. 7 and 8 containing at least a test strip can be used for the detection of clostridium presented in a liquid sample. Add 250 microliters of the sample to a collection device such as a test tube, followed by adding 100 microliters of 500 millimolar sodium phosphate buffer, pH 7.4, containing 9 grams/liter sodium chloride, 1 gram/liter bovine serum albumin and 5 milligrams/liter EDTA. Allow this mixed solution to incubate for 30 seconds. Then approximately 300 microliters of the liquid sample is added into the specimen container of the present invention. At least a portion of the liquid sample (approximately 160 microliters) will enter into the test chamber through a conduit. Sample flow is initiated on the test strip by capillary action and the result of the test can be viewed through the test result window within about 10 minutes after the test is initiated. A control line is preferably present to indicate that proper flow has occurred. The rest of the liquid sample in the container can be used for confirmatory test if needed.

What is claimed is:

1. A collecting and testing device, comprising:
a) a specimen container for receiving a specimen, said container comprising (i) an open proximal end through which the specimen is added to the container, and (ii) a distal end;
b) a testing chamber comprising at least a test element; and
c) a conduit comprising two openings that conveys a portion of the specimen from the specimen container to the testing chamber so that a portion of the specimen contacts the test element in the testing chamber,
wherein the testing chamber and the specimen container are in indirect fluid communication, the specimen in the specimen container is separable from the specimen in the testing chamber, and the specimen in the testing chamber cannot flow back into the specimen container.

2. The device of claim 1, further comprising a cover for covering the open proximal end of the specimen container, and the specimen container has a coupling structure in the top portion of the container for receiving said cover with a mating structure.

3. The device of claim 1, wherein the specimen container is substantially cylindrical.

4. The device of claim 1, wherein the testing chamber comprises a proximal end, a distal end, a front wall, and a back wall.

5. The device of claim 4, wherein one opening of the conduit is proximal to a distal end of the specimen container for receiving a portion of the specimen from said container, and another opening of the conduit is proximal to a lower end of the back wall of the testing chamber for sending the specimen into the testing chamber.

6. The device of claim 1, wherein the conduit comprises one or more filters that allow a portion of the specimen to flow into the testing chamber, but prevent the specimen from flowing back from the testing chamber to the specimen container.

7. The device of claim 1, wherein the testing chamber comprises one or more windows for viewing a test result in the testing chamber.

8. The device of claim 7, wherein the testing chamber has a flat surface so that said test result in the testing chamber can be photocopied or scanned.

9. The device of claim 1, wherein the testing chamber is capable of trapping air or gas that helps to maintain the specimen to a desirable level in the testing chamber and to prevent the testing chamber from being overloaded with the specimen.

10. The device of claim 1, wherein the test element comprises one or more test strips.

11. The device of claim 10, wherein the test strip comprises a sample application zone, a reagent zone and a test result zone.

12. A method of detecting an analyte in a specimen, the method comprises the steps of:
a. providing a specimen comprising an analyte;
b. applying the specimen to the specimen container of the device of claim 1;
c. automatically transferring a portion of the specimen from the specimen container to the testing chamber of the device of claim 1 through the conduit of the device of claim 1;
d. contacting the specimen with one or more test elements in the testing chamber; and
e. detecting the analyte in the specimen by the test element in the testing chamber.

13. The method of claim 12, wherein the specimen comprises a biological fluid.

14. The method of claim 12, wherein the analyte is selected from the group consisting of a hormone, a drug, a protein, and an etiological agent.

15. The method of claim 12, wherein the specimen is extracted in the specimen container.
